(19) United States
(12) Patent Application Publication
(10) Pub. No.: US 2014/0316231 A1
(43) Pub. Date: Oct. 23, 2014

(54) ECG ELECTRODE FOR USE IN X-RAY ENVIRONMENTS

(71) Applicant: KONINKLIJKE PHILIPS N.V., EINDHOVEN (NL)

(72) Inventors: Randall Peter Luhta, Chardon, OH (US); Allan Joseph Peusek, Mentor, OH (US); Brandon Keller Richards, Hudson, OH (US); James Thomas Richards, Willoughby Hills, OH (US); David Dennis Salk, Parma, OH (US)

(21) Appl. No.: 14/355,666

(22) PCT Filed: Nov. 13, 2012
(86) PCT No.: PCT/IB2012/056389

§ 371 Date: May 1, 2014

Related U.S. Application Data
(60) Provisional application No. 61/562,489, filed on Nov. 22, 2011.

(51) Int. Cl. A61B 5/0416; A61B 6/00

(52) U.S. Cl. CPC A61B 5/0416; A61B 6/541
USPC 600/394; 600/395; 29/874

(57) ABSTRACT

An ECG electrode is provided which can be placed within the direct path of x-rays during an imaging scan without inducing an x-ray induced erroneous current. The ECG electrode has a support element with a conductive post on one side electrically connected to a conductive plate on the other side. A dissipative anti-static element in or near the ECG electrode dissipates static electricity which forms on the surfaces of the insulating components in the ECG electrode. The dissipative anti-static element may be, for example, a slightly conductive property of the bulk material used to make the insulating material, or a conductive coating added to the insulating material surfaces. The dissipative anti-static element may also be incorporated in the clamp attached to the conductive post. In a further embodiment, an ion blower aimed at the ECG electrode may be used to remove static electricity.

The present application relates generally to the imaging arts and more particularly to an ECG electrode for use in x-ray environments. The application subject matter finds particular use in connection with x-ray based imaging systems such as for example general radiography, x-ray computed tomography (CT), fluoroscopic or real-time x-ray imaging, x-ray based angiography, and the like.

X-ray based imaging systems are widely used in the medical field, the security field, and other fields. These imaging systems generate x-rays which pass through an object, such as a human person, and then record the attenuated x-rays after they pass through the object to generate imaging data for later analysis and use. Such uses include for example medical diagnosis and treatment, looking for illegal or dangerous items such as guns and knives for security purposes, and the like. Thus, while one embodiment is medical imaging and much of the following description relates to the medical imaging field, the present invention applies in other fields as well. It also applies in other non-imaging environments where x-rays are employed in combination with ECG electrodes.

In many contexts, it is desirable to monitor a patient's heart beat during an x-ray based imaging scan. The monitored heart beat data can be combined with the imaging data recorded by the imaging system in many ways. For example, when the heart is one of the organs being imaged by the system, the system can synchronize the heart beat data and the imaging data in time so that health care professionals will know what phase of the cardiac cycle is being imaged. Historically, such methods were first applied retroactively. That is, heart beat data and imaging data were simultaneously recorded and then, at a later time, both sets of data are processed by the imaging system to synchronize the imaging data of interest to health care professionals.

More recently, such methods have also been applied proactively, including for example “step and shoot” implementations. In these proactive methods, the heart beat data is monitored and is used to trigger or “gate” imaging scans of the heart, so that imaging data is generated only for the portion(s) of the cardiac cycle which are of interest to health care professionals. When a beginning of an appropriate portion of the cardiac cycle is detected, the x-ray beam is turned on and the imaging data acquisition system collects the x-ray attenuation data required to make an image. For example, in many cases, imaging data only of the cardiac rest phase is desired. Proactive imaging techniques can minimize the patient's x-ray exposure by ensuring that the x-ray source is turned off during more active cardiac phases, which are not necessarily of interest.

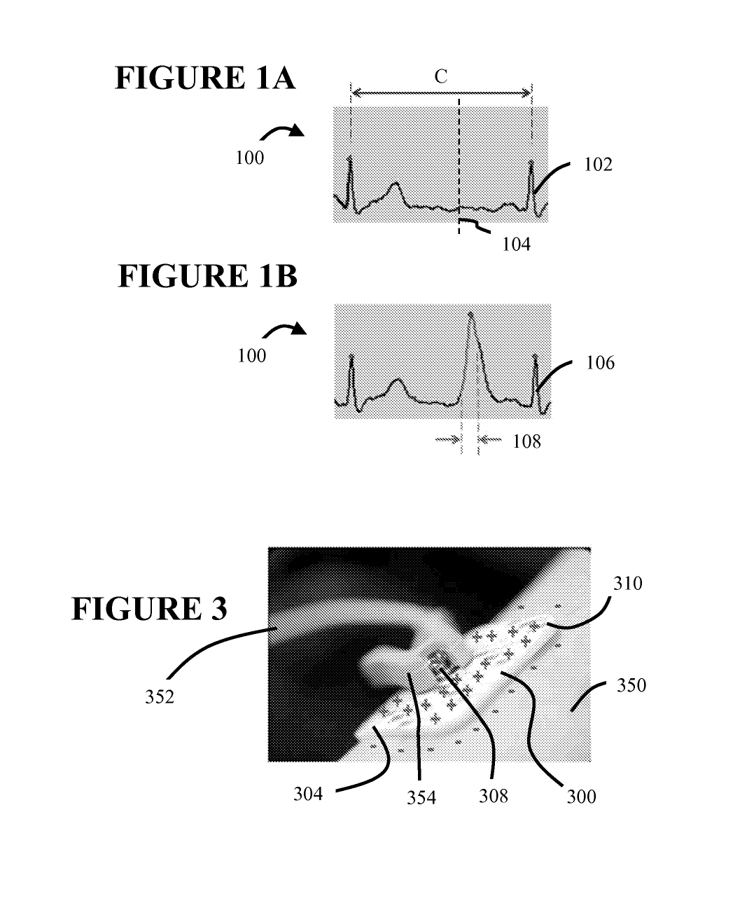

Electrocardiography (ECG or EKG) is a technique commonly used to monitor a patient's heart beat in many different contexts, including medical imaging. Employing this technique, electrodes are attached to the outer surface of the patient's skin in order to monitor the electrical activity of the heart. The electrodes are connected by lead wires to an external device, which records the electrical activity of the heart over a period of time as detected by the electrodes. The data recording produced by the ECG technique is an electrocardiogram. FIG. 1A illustrates a typical electrocardiogram 100 recording of a normal ECG data signal 102, where the horizontal axis represents time and the vertical axis represents electrical activity. The time period identified as “C” reflects one cardiac cycle. In the normal condition of FIG. 1A, if the horizontal time axis were extended to the right, the same electrical activity cycle “C” would be repeated over time. Thus, using standard techniques, an imaging system may employ ECG data such as the normal signal 102 to trigger imaging scans at appropriate points during the cardiac cycle C, such as the trigger point 104.

However, if the x-rays which are used to generate the imaging data are permitted to interact with the ECG electrodes, difficulties can arise. More particularly, the imaging x-rays often generate an erroneous signal current in the ECG device. FIG. 1B illustrates a typical electrocardiogram 100 recording of a disrupted ECG data signal 106, including an x-ray induced erroneous signal current 108. This erroneous signal current 108 can disrupt the ability of the imaging scanner to synchronize to the proper phase of the cardiac cycle and can cause an imaging scan to abort. This in turn results in a need to re-scan the patient, causing an extra x-ray dose to the patient.

Conventionally, to avoid this problem, imaging system manufacturers have instructed users to place the ECG electrodes outside of the x-ray path during a cardiac imaging scan—for example on the patient's shoulder or belly. These locations are close enough to the heart to generate a satisfactory record of the heart's electrical activity for imaging purposes, while at the same time being far enough away from the heart to avoid any x-ray induced erroneous current 108. Unfortunately, in other non-imaging contexts where ECG is employed, it is customary to place the ECG electrodes very close to the heart. Health care professionals are prone to follow the same procedure out of habit during x-ray based imaging, despite instructions to the contrary which accompany the imaging system. Also, in other cases, the imaged patient may already have electrodes placed in a standard close-to-heart position for general heart monitoring prior to the imaging scan, which it would be inconvenient to move or replace. Thus x-ray induced erroneous currents 108 can be generated, leading to poor imaging results.

According to one aspect of the present invention, an ECG electrode is provided which can be placed within the direct path of x-rays during an imaging scan without inducing an x-ray induced erroneous current. The ECG electrode employs materials that eliminate or reduce static electricity forming on the insulating material surfaces of the electrode. In one aspect, the insulating materials may be “dissipative” to have a slight electrical conductivity so that they dissipate static electricity but do not interfere with the ECG electrode's monitoring of the heart beat. This dissipative anti-static conductivity can, for example, result from a bulk property of the materials used to construct the insulating materials of the electrode, or from a conductive coating added to those material surfaces. Numerous advantages and benefits will become apparent to those of ordinary skill in the art upon reading the following detailed description of several embodiments. The invention may take form in various components and arrangements of components, and in various process operations and arrangements of process operations. The drawings are only for the purpose of illustrating many embodiments and are not to be construed as limiting the invention.

FIG. 1A illustrates a typical ECG data signal under normal conditions, including electrical activity over one cardiac cycle C;

FIG. 1B illustrates a disrupted ECG data signal which may occur when the ECG electrodes are exposed to x-ray which cause an x-ray induced erroneous signal current;

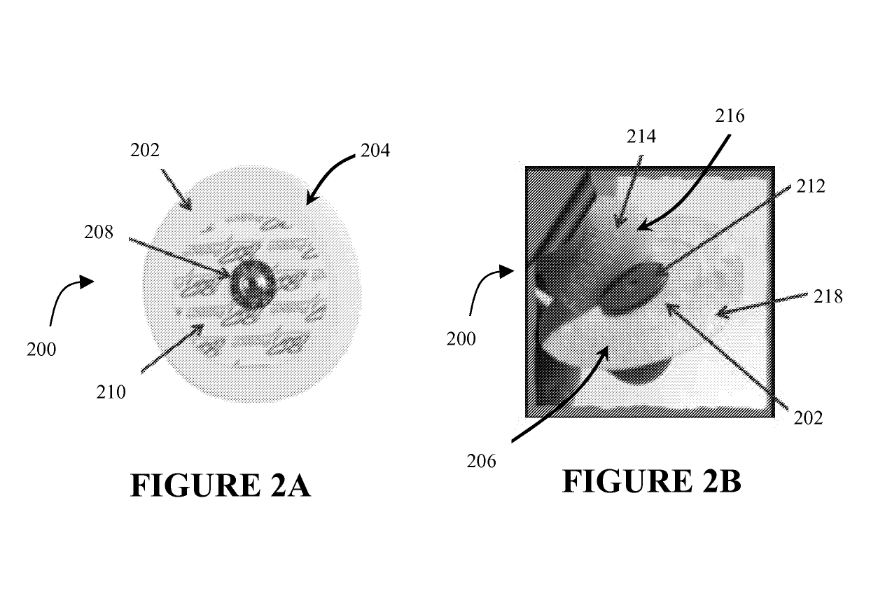

FIG. 2A is a wire-side perspective view of an exemplary ECG electrode;

FIG. 2B is a patient-side perspective view of the ECG electrode in FIG. 1A, in a state of partial disassembly;

FIG. 3 shows an ECG electrode attached to a patient, and illustrates how it is believed that an x-ray induced erroneous signal current might be formed; and

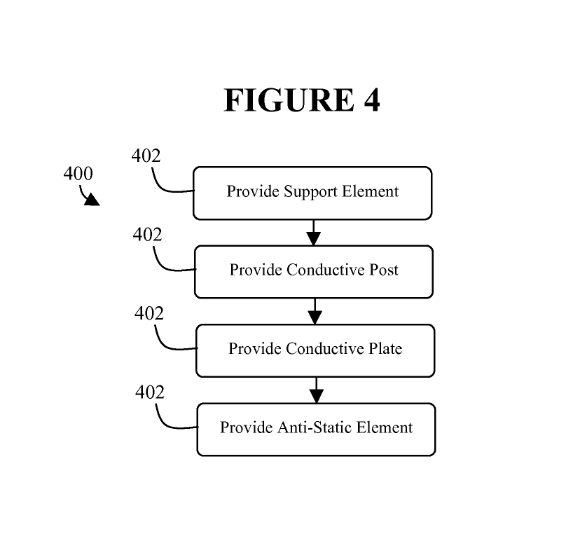

FIG. 4 is an illustration of an exemplary method for making an ECG electrode with dissipative anti-static properties.

The subject matter of the present disclosure finds use in connection with any imaging system in which an imaged object, such as a human patient, is concurrently exposed to x-rays and electrically monitored by an ECG unit. ECG electrodes are manufactured using many different sizes, shapes, materials and constructions. A typical ECG electrode 200, such as a pre-gelled Ag/AgCl type electrode, is illustrated in FIGS. 2A and 2B. The electrode 200 includes an insulating support element 202 having an outer side 204 to which a lead wire is attached and an inner side 206 which adheres to the patient. The support element 202 is typically composed of an insulating foam material, such as a polyethylene foam.

The outer side 204 of the support element 202 has a conductive post or stud 208. The post 208 is typically made of a sturdy metal, but any electrically conductive material may be used. An ECG lead wire may be removably attached to the conductive post 208 by a small clamp, clip, or other connecting mechanism. A label 210 is often located on the outer side 204 of the electrode 200, and is typically made of an insulating plastic material.

The inner side 206 of the support element 202 has a conductive plate 212. In the common Ag/AgCl type electrode, the conductive plate is made of silver (Ag). Other conductive materials may be used, however, such as carbon. A sponge 214 containing an electrically conductive gel 216, such as a silver chloride (AgCl) gel, may cover the conductive plate 212. The inner side 206 of the electrode 200 is coated with or contains an adhesive 218 so that, when it is placed against a patient's skin, the electrode 200 adheres to the patient. When the electrode 200 is placed on the patient, the gel 216 forms a conductive path from the patient's skin to the conductive plate 212, which then leads to the conductive post 208 on the other side 204 of the electrode 200.

As will be appreciated, the electrode 200 includes both electrically conducting and electrically insulating materials. The sponge 214 soaked with an electrically conductive gel 216, the conductive plate 212, and the conductive post 208 are all conducting materials, designed to carry electrical signals which are indicative of the patient's heart beat. The support element 202 and the label 210 are insulating materials, designed to provide structure and easy handling to hold the conducting materials in place so they may perform that function.

The insulating materials of an ECG electrode—such as the support element 202 and the label 210 of the exemplary electrode 200—are capable of holding a large amount of static electricity on their surfaces. Static electricity potentials of between about 10 and about 1000 Volts are not uncommon. In most contexts where ECG is employed, this does not cause a problem. However, in the specific context of x-ray based imaging systems and perhaps other contexts, it is believed that this static electricity potential can interfere with the operation of the ECG electrode.

This is shown, for example, in FIG. 3. An ECG electrode 300 is adhered to the skin of a patient 350. A lead wire 352 has a clamp 354 which is attached to the conductive post 308 of the electrode 300. The outer side 304 of the electrode 300 has a plastic label 310 which has a positive potential (+) of static electrical energy present on its outer surface. That positive potential attracts negatively charged ions (−) in the air around the plastic label 310. It is believed that in normal circumstances, the air acts as an insulator, so that negatively charged ions in the air do not have an electrical path into the electrical components of the electrodes 300 or the skin of the patient. However, when the x-rays of the imaging scanner pass through the air near to the electrode 300, it is believed that many positive and negative air ions are formed in the vicinity of the electrode 300. It is believed that this large mixture of positive and negative air ions creates electrical discharge paths from the statically charged insulating material surface of the label 310 to the conductive post 308 and to the patient's skin, thereby generating an erroneous signal current in the ECG device such as illustrated in FIG. 1B. Of course, in other cases the potential of static electrical energy on the outer insulating material surfaces of the electrode may be negative, but it is believed that essentially the same process occurs to generate an erroneous signal current in the ECG electrode 300. The build-up of static electrical energy on insulating surfaces of an ECG electrode may be eliminated using any one of several methods.

In a first embodiment, a dissipative anti-static element may be provided by composing the insulating materials of bulk materials which, while having a high resistance to electricity, are nonetheless slightly conductive. That is, the bulk materials have an electrical resistance that is low enough to dissipate static electricity via the conductive post, the conductive plate, and/or conductive gel, before it can significantly build up. At the same time, however, the bulk material electrical resistance is high enough not to impede with the normal functioning of the electrode. Conductive foams and plastic are known. It is believed that a bulk or volume resistivity of from about 10⁴ Ω-cm to about 10¹¹ Ω-cm is appropriate for most ECG electrode insulating materials.

In a second embodiment, a dissipative anti-static element comprises coating the surfaces of the insulting materials with a conducting material which allows static charge to bleed away to the conductive post. This coating may be a liquid or a solid in form, although most commonly it is applied as a liquid which dries to become a solid coating. Suitable liquid dissipative anti-static coatings, for example, are generally known to protect sensitive electronic components from electrostatic discharge (ESD). Suitable solid dissipative anti-static coatings include slightly conductive paper, slightly conductive plastic, slightly conductive rubber, laminates with at least one slightly conductive layer, and materials with a low propensity for triboelectric charging. It is believed that a surface resistance of from about 10⁵ Ω/sq to about 10¹² Ω/sq, or from 10⁷ Ω/sq to about 10¹² Ω/sq, is appropriate for most dissipative anti-static coatings on an ECG electrode. As will be appreciated by one of ordinary skill, these units are stated in ohms (Ω) per a unitless measure of area (sq).

In a third embodiment, a dissipative anti-static element may be incorporated in the clamp which is attached to the conductive post of the electrode, such as the clamp 354 attached to the electrode 300 in FIG. 3. Many imaging scanners used for cardiac imaging include an integral or dedicated ECG unit, with a permanent “harness” incorporating lead wires and clamps. Each time a patient is scanned in conjunction with recording ECG data, a new electrode is adhered to the patient and then discarded. The clamp of the ECG harness may include a dissipative anti-static element which contacts all the insulating materials of an ECG electrode when connected thereto, to dissipate any build up of static electricity.

In a fourth embodiment, a balanced stream of compressed ionized air is created and directed on to the insulating material surfaces, to remove the static electricity from the surfaces. Ionizing blowers such as blow-off guns are commercially available.

Yet further embodiments made include combining one or more of the foregoing embodiments. For example, the insulating materials of the electrode may be composed of bulk materials which are conductive enough to dissipate static electricity, and also additionally have surfaces which are coated with a conducting material.

Another way of measuring the electrical resistance of an insulating material is discharge time. In any of the foregoing embodiments, the dissipative anti-static element may have a discharge time of from about 0.01 second to about 30 seconds.

An exemplary method 400 for making an ECG electrode with dissipative anti-static properties is illustrated in FIG. 4. The method 400 includes providing 402 a support element comprising an insulating material and having an outer side (204) and an inner side (206) opposite the inner side. The method further includes providing 404 a conductive post on the outer side of the ECG electrode, and providing 406 a conductive plate on the inner side of the support element which is electrically connected to the conductive post. The method also includes providing 408 a dissipative anti-static element to dissipate static electricity which forms on the surfaces of the insulating components in the ECG electrode. The method may include several other additional steps, such as providing any of the elements described above, and the steps may be performed in any convenient order.

The invention has been described with reference to the several embodiments. Obviously, modifications and alterations will occur to others upon reading and understanding the preceding detailed description. It is intended that the invention be construed as including all such modifications and alterations insofar as they come within the scope of the appended claims or the equivalents thereof. The invention may take form in various compositions, components and arrangements, combinations and sub-combinations of the elements of the disclosed embodiments.

As one example, while the present description focuses on combining x-ray based imaging with ECG monitoring, it also applies to other non-imaging environments.

1. An ECG electrode comprising:
a support element comprising an insulating material and having an outer side and an inner side opposite the outer side;
a conductive post disposed on the outer side of the ECG electrode;
a conductive plate disposed on the inner side of the support element, and electrically connected to the conductive post; and
a dissipative anti static element to dissipate static electricity which forms on the surfaces of the insulating components in the ECG electrode.

2. The ECG electrode of claim 1, wherein the support element comprises a bulk material which incorporates the dissipative anti-static element.

3. The ECG electrode of claim 2, wherein the support element comprises a conductive foam or plastic.

4. The ECG electrode of claim 2, wherein the bulk material has a bulk resistivity of from about 10⁴ Ω-cm to about 10¹¹ Ω-cm.

5. The ECG electrode of claim 1, wherein the dissipative anti-static element comprises a conductive material coating on the surfaces of the insulating components.

6. The ECG electrode of claim 5, wherein the coating has a surface resistance of from about 10⁵ Ω/sq also to about 10² Ω/sq.

7. An imaging scanner system comprising:
an ECG electrode comprising a support element, a conductive post and a conductive plate, wherein the support element comprises an insulating material and has an outer side and an inner side opposite the outer side, and the conductive post is disposed on the outer side of the electrode, and the conductive plate is disposed on the inner side of the support element and is electrically connected to the conductive post; and
a dissipative anti-static element to dissipate static electricity which forms on the surfaces of the insulating components in the ECG electrode.

8. The imaging scanner system of claim 7, wherein the support element comprises a bulk material which incorporates the dissipative anti-static element.

9. The imaging scanner system of claim 8, wherein the support element comprises a conductive foam or plastic.

10. The imaging scanner system of claim 8, wherein the bulk material has a hulk resistivity of from about 10⁴Ω-cm to about 10¹¹ Ω-cm.

11. The imaging scanner system of claim 7, wherein the dissipative anti-static element comprises a conductive material coating on the surfaces of the insulating components.

12. The imaging scanner system of claim 11, wherein the coating has a surface resistance of from about 10⁵ Ω/sq also to about 10¹² Ω/sq.

13. The imaging scanner of claim 7, further comprising an ECG lead wire including a clamp comprising the dissipative anti-static element in contact with the insulating materials of the ECG electrode to dissipate the static electricity.

14. The imaging scanner of claim 7, wherein the dissipative anti-static element comprises an ion blower aimed at the ECG electrode.

15. A method of manufacturing an ECG electrode, the method comprising:
providing a support element comprising an insulating material and having an outer side and an inner side opposite the outer side;
providing a conductive post on the outer side of the ECG electrode;
providing a conductive plate on the inner side of the support element, and electrically connected to the conductive post; and
providing a dissipative anti-static element to dissipate static electricity which forms on the surfaces of the insulating components in the ECG electrode.

16. The method of claim 15, wherein the support element comprises a hulk material which incorporates the dissipative anti-static element.

17. The method of claim 16, wherein the support element has a bulk resistivity of from about 10⁴ Ω-cm to about 10¹¹ Ω-cm.

18. The method of claim 15, wherein method further comprises placing a dissipative anti-static conductive material coating on the surfaces of the insulating components.

19. The method of claim 18, wherein the coating has a surface resistance of from about
10⁵ Ω/sq to about 10⁵ Ω/sq.

20. An ECG electrode clamp comprising a dissipative anti-static element to contact an insulating material of an ECG electrode to dissipate static electricity which forms on the surfaces of the insulating material in the ECG electrode.

21. The ECG electrode clamp of claim 20, wherein the dissipative anti-static element comprises a bulk material of the clamp.

22. The ECG electrode damp of claim 20, wherein the dissipative anti-static element comprises a conductive material coating disposed on the damp.
